SEQUENCE LISTING (10 sequences)

SEQ ID NO: 1 - DNA, 21 bp, synthetic construct
  source 1..21: mol_type = other DNA
aggtcggtgtgaacggatttg

SEQ ID NO: 2 - DNA, 23 bp, synthetic construct
  source 1..23: mol_type = other DNA
tgtagaccatgtagttgaggtca

SEQ ID NO: 3 - DNA, 22 bp, synthetic construct
  source 1..22: mol_type = other DNA
gcaactgttcctgaactcaact

SEQ ID NO: 4 - DNA, 21 bp, synthetic construct
  source 1..21: mol_type = other DNA
atcttttggggtccgtcaact

SEQ ID NO: 5 - DNA, 20 bp, synthetic construct
  source 1..20: mol_type = other DNA
tggggctcttcaaaagctcc

SEQ ID NO: 6 - DNA, 23 bp, synthetic construct
  source 1..23: mol_type = other DNA
aggaactatcaccggatcttcaa

SEQ ID NO: 7 - DNA, 21 bp, synthetic construct
  source 1..21: mol_type = other DNA
cagctccaagaaaggacgaac

SEQ ID NO: 8 - DNA, 21 bp, synthetic construct
  source 1..21: mol_type = other DNA
ggcagtgtaactcttctgcat

SEQ ID NO: 9 - DNA, 21 bp, synthetic construct
  source 1..21: mol_type = other DNA
ccaagtgctgccgtcattttc

SEQ ID NO: 10 - DNA, 21 bp, synthetic construct
  source 1..21: mol_type = other DNA
ggctcgcagggatgatttcaa

(19) United States
(12) Patent Application Publication
(10) Pub. No.: US 2025/0302989 A1
(43) Pub. Date: Oct. 2, 2025

(54) STING AGONIST-CONTAINING UREASE-POWERED NANOMOTOR-BASED BLADDER CANCER IMMUNOTHERAPY AGENT

(71) Applicant: PHI BIOMED INC., Seoul (KR)

(72) Inventors: Hyun Sik CHOI, Changwon-si (KR); Seung-Hwan JEONG, Seoul (KR)

(21) Appl. No.: 18/832,442

(22) PCT Filed: Jan. 25, 2023
(86) PCT No.: PCT/KR2023/001103

§ 371 Date: Jul. 23, 2024

(30) Foreign Application Priority Data
Jan. 24, 2022 (KR) 10-2022-0010085

(51) Int. Cl. A61K 47/69; A61K 9/00; A61K 31/282; A61K 31/337; A61K 31/407; A61K 31/513; A61K 31/519; A61K 31/704; A61K 31/722; A61K 31/727; A61K 33/243; A61K 38/12; A61K 38/14; A61K 38/39; A61K 38/50; B82Y 5/00

(52) U.S. Cl. CPC A61K 47/6949; A61K 9/0004; A61K 31/282; A61K 31/337; A61K 31/407; A61K 31/513; A61K 31/519; A61K 31/704; A61K 31/722; A61K 31/727; A61K 33/243; A61K 38/12; A61K 38/14; A61K 38/39; A61K 38/50; B82Y 5/00

(57) ABSTRACT

A chitosan-heparin nanomotor and a method for producing same are disclosed. A STING agonist-encapsulated urease-based chitosan-heparin nanomotor delivers the STING agonist directly to bladder mucosal cells in the bladder, and thus can induce an immune response.

### SEQUENCE LISTING

The instant application contains a Sequence Listing which has been submitted electronically in XML file format and is hereby incorporated by reference in its entirety. Said XML copy, created on Apr. 9, 2025, is named DANA-109-PCT-US_SL.xml and is 9,876 bytes in size.

### CROSS-REFERENCE TO RELATED APPLICATIONS

This application is the U.S. National Stage entry of International Application No. PCT/KR2023/001103, filed on Jan. 25, 2023, which, in turn, claims priority to Korean Patent Application No. 10-2022-0010085, filed on Jan. 24, 2022, both of which are hereby incorporated herein by reference in their entireties for all purposes.

### TECHNICAL FIELD

The present invention relates to a method for producing a STING agonist-encapsulated urease-powered nanomotor-based bladder cancer immunotherapy agent and a use thereof.

### BACKGROUND ART

Bladder cancer is the 9^{th} most common cancer worldwide, with 430,000 new cases diagnosed each year and the mortality rate associated with bladder cancer is the 13′ highest. Bladder cancer occurs more frequently in men and is the 4^{th} most common cancer among men in the United States. According to domestic reports, bladder cancer is an important cancer type that has the 10^{th} highest incidence among newly diagnosed cancer cases in men in 2017 and exhibits the 10^{th} highest cancer-related mortality rate in 2019.

Since bladder cancer is accompanied by symptoms of hematuria, 75 to 80% of cases are found to be non-muscle invasive bladder cancer. Among these cases, when bladder cancer is in the Ta stage with high-grade differentiation, or is in the T1 stage or Tis, transurethral resection of bladder tumors is performed followed by intravesical Bacille Calmette-Guerin (BCG) injection therapy. Nevertheless, approximately 30% of patients progress to muscle-invasive bladder cancer, reaching a stage requiring a radical cystectomy of entire bladder. While the 5-year survival rate for non-muscle-invasive bladder cancer is over 70%, the 5-year survival rate for muscle invasive bladder cancer is 36%. For cases accompanied by metastasis, the 5-year survival rate is less than 10% with an extremely poor prognosis. Furthermore, a radical cystectomy of entire bladder performed for the treatment of muscle-invasive bladder cancer may cause complications such as intestinal obstruction, urinary tract infection, surgical site hernias, and decrease quality of life in patients after surgery.

STING recognizes cyclic dinucleotides (CDNs) to activate the type I pathway, thereby increasing the innate immune response and thus enhancing the adaptive T cell response. As a representative STING agonist, cyclic GMP-AMP (cGAMP) is synthesized from ATP and GTP by recognizing DNA that has entered into the cytoplasm. cGAMP may be degraded by ENPP1 that is a type II transmembrane glycoprotein, and exhibits no sufficient effect in systemic therapy. Meanwhile, cGAMP exhibits an anti-tumor effect by intertumoral injection. Thus, it is most important for the treatment of cancer using STING agonist to increase the retention time in the target tissue while suppressing degradation of STING agonist. In this regard, when a drug is directly injected into the bladder, the bladder may absorb the drug through the process of endocytosis, which may be a suitable target for STING agonist treatment when an appropriate delivery system exists. However, the function of the STING agonist may be reduced when the STING agonist is exposed to urine for a long time, and because the bladder mucosa is covered with a glycosaminoglycan layer, the ability to deliver the drug to cells by improving the permeability through the glycosaminoglycan layer has become an important issue.

Therefore, the present inventors propose a urease-powered nanomotor, specifically a urease-powered nanomotor capable of delivering a STING agonist. The urease-powered nanomotor has a structure in which urease is combined with a chitosan-heparin nanocomplex or PLGA nanoparticles having good mucosal adhesiveness. It is anew drug delivery platform that can not only block the deterioration of the drug function in urine, but also enhance the efficiency of delivery to bladder cells through the mucosa using the propulsive force of the nanomotor. It is expected that STING activation in bladder cancer tissue can be maximized through the synthesis of the STING agonist and the urease-powered nanomotor.

### DISCLOSURE

### Technical Problem

The present invention is directed to providing a biocompatible nanomotor capable of penetrating through the bladder wall under biological conditions to remain in the bladder for a long time.

Also, the present invention is directed to providing a nanomotor capable of inducing an immune response to treat bladder diseases by directly delivering a STING agonist to bladder mucosal cells in the bladder.

### Technical Solution

The present invention provides a biocompatible polymer nanomotor, which includes:
biocompatible polymer nanoparticles; and
urease bound to the surfaces of the biocompatible polymer nanoparticles,
wherein the biocompatible polymer nanoparticles include one or more selected from the group consisting of chitosan, heparin, and poly(lactide-co-glycolide) (PLGA), and
the urease generates gas in the presence of urea to induce self-propulsion of the nanomotor.

Also, the present invention provides a method for producing a biocompatible polymer nanomotor, which includes: producing urease-bound biocompatible polymer nanoparticles by binding urease to the surfaces of biocompatible polymer nanoparticles,
wherein the biocompatible polymer nanoparticles include one or more selected from the group consisting of chitosan, heparin, and poly(lactide-co-glycolide) (PLGA).

Further, the present invention provides a carrier for a drug delivery system including the above-described biocompatible polymer nanomotor.

### Advantageous Effects

A biocompatible polymer nanomotor according to the present invention can treat bladder cancer by penetrating deeply through the mucosal layer of the bladder to remain in the bladder wall for a long time.

In particular, the chitosan-heparin nanomotor moves autonomously in the presence of urea, and after reaching the bladder wall, the chitosan-heparin nanomotor is effectively attached to the mucosal layer due to chitosan on the surface of the nanomotor.

After injection into the bladder, the biocompatible polymer nanomotor can penetrate deep into bladder tissue by self-propulsion, and thus a large amount of the nanomotor can be retained in the bladder even after urination. Through this, the efficiency of drug delivery in the bladder is excellent and the therapeutic effect can be maximized.

### DESCRIPTION OF DRAWINGS

FIG. 1A is a schematic diagram showing a process of synthesizing a STING agonist-encapsulated chitosan-heparin nanomotor, and FIG. 1B is a schematic diagram showing a process of treating bladder cancer.

FIG. 2A shows the encapsulating efficiency of a STING agonist into a chitosan-heparin nanocomplex, and FIG. 2B shows the zeta potential of the nanocomplex after encapsulating the STING agonist. FIG. 2C shows a TEM image of the STING agonist-encapsulated chitosan-heparin nanomotor, and FIG. 2D shows the size of the nanomotor in an aqueous solution. Also, FIG. 2E shows the zeta potential of the STING agonist-encapsulated chitosan-heparin nanocomplex before and after attachment of urease, and FIG. 2F shows the results of comparing the activity efficiency of urease attached to the nanocomplex to that of urease which is not attached to the nanocomplex. In addition, FIG. 2G shows the absorbance of the STING agonist-encapsulated chitosan-heparin nanocomplex and the nanomotor, and FIG. 2H shows the release experiment results of the STING agonist in neutral and slightly acidic conditions.

FIG. 3A is a fluorescence image of dendritic cells, showing the inflow amount of the nanomotor when the dendritic cells are incubated with a fluorescently labeled STING agonist-encapsulated chitosan-heparin nanomotor for 2 hours. FIGS. 3B and 3C show the mechanism for inflow of the STING agonist-encapsulated chitosan-heparin nanomotor into dendritic cells, and FIGS. 3D and 3E show the activation of dendritic cells by the STING agonist-encapsulated chitosan-heparin nanomotor.

FIG. 4A shows the mean square displacement (MSD) value of the STING agonist-encapsulated chitosan-heparin nanomotor according to the concentration (0, 50, 100, and 200 mM) of urea, FIG. 4B shows the tracking line and speed of the STING agonist-encapsulated chitosan-heparin nanomotor, and FIG. 4C shows the diffusion area of the STING agonist-encapsulated chitosan-heparin nanomotor cluster for 90 seconds.

FIG. 5A shows a schematic diagram of bio-imaging to confirm penetration and retention efficacy in the bladder. FIG. 5B shows the results of comparing the bladder penetration depths of the chitosan-heparin nanocomplex and the chitosan-heparin nanomotor for 120 minutes using a two-photon microscope, and FIGS. 5C and 5D show the fluorescence intensities at 0 and 120 minutes, respectively. Also, FIG. 5E shows the bladder fluorescence image 12 hours after injection of the chitosan-heparin nanocomplex and the chitosan-heparin nanomotor, FIG. 5F shows an IVIS image, and FIG. 5G shows the fluorescence intensity.

FIG. 6A shows a schedule for the production and treatment of a bladder cancer model, and FIG. 6B shows a representative bladder tissue image after 2 weeks. Also, FIGS. 6C and 6D show the results of comparison of bladder cancer thickness and number of T cells in the bladder, respectively (confirmed the bladder cancer therapeutic efficacy), and FIG. 6E shows the results of comparison of immune-related mRNA expression.

### BEST MODE

The present invention relates to a biocompatible polymer nanomotor, which includes:
biocompatible polymer nanoparticles; and
urease bound to the surfaces of the biocompatible polymer nanoparticles,
wherein the urease generates gas in the presence of urea to induce self-propulsion of the nanomotor.

In the present invention, the structure in which urease is bound to the biocompatible polymer nanoparticles may be expressed as a biocompatible polymer nanomotor. Also, the structure in which a STING agonist is encapsulated into the interior of biocompatible polymer nanoparticles may be expressed as STING agonist-encapsulated biocompatible polymer nanoparticles, and the structure in which urease is bound to the STING agonist-encapsulated biocompatible polymer nanoparticles may be expressed as a STING agonist-encapsulated biocompatible polymer nanomotor.

For example, when a chitosan-heparin nanocomplex is used as a biocompatible polymer nanoparticle, the structure in which urease is bound to the chitosan-heparin nanocomplex may be expressed as a chitosan-heparin nanomotor. Also, the structure in which the STING agonist is encapsulated into the interior of the chitosan-heparin nanocomplex may be expressed as a STING agonist-encapsulated chitosan-heparin nanocomplex (STING@nanocomplex), and the structure in which urease is bound to the STING agonist-encapsulated chitosan-heparin nanocomplex may be expressed as a STING agonist-encapsulated chitosan-heparin nanomotor (STING@nanomotor).

Hereinafter, the biocompatible polymer nanomotor of the present invention will be described in more detail.

In the present invention, the term “nanomotor” refers to a nanoparticle that may be propelled with a force applied by various external stimuli, and is defined as a microscopic device that has its own propulsion through the chemical reaction of a catalyst in a liquid. These nanomotors may maintain self-propulsion in a liquid and contribute to solving complex and difficult problems while being given a mission.

The biocompatible polymer nanomotor according to the present invention includes:
biocompatible polymer nanoparticles; and
urease bound to the surfaces of the biocompatible polymer nanoparticles.

In the present invention, because the biocompatible polymer nanoparticles have excellent mucosal adhesiveness, the biocompatible polymer nanoparticles can enhance the efficiency of drug delivery to bladder cells through the mucosa.

According to one exemplary embodiment, the biocompatible polymer nanoparticles may include one or more selected from the group consisting of chitosan, heparin, and poly(lactide-co-glycolide) (PLGA).

In the present invention, chitosan is a natural polymer that has an aminopolysaccharide structure and cationic properties, and includes repeating monomer units of Chemical Formula 1:

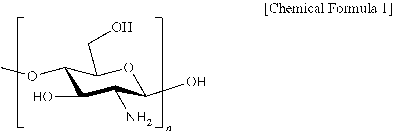

wherein n is an integer and represents a degree of polymerization. That is, it represents the number of monomer units in the chitosan chain.

The chitosan generally contains a proportion of monomer units in which an amino group is acetylated. In fact, chitosan is obtained by deacetylation of chitin (100% acetylated). The degree of deacetylation may generally be in the range of 30 to 95, preferably 55 to 90, which indicates that 10% to 45% of amino groups are acetylated.

According to one exemplary embodiment, chitosan may have a molecular weight of 50 to 190 kDa, preferably 20 to 100 kDa, or 50 to 150 kDa.

In the present invention, heparin is a natural material in the blood and is a polysaccharide involved in the blood coagulation process.

According to one exemplary embodiment, heparin may have a molecular weight of 17 to 19 kDa.

In the present invention, PLGA is a polymer manufactured by synthesizing lactide (LA) and glycolide (GA). In this case, the decomposition rate and physical properties may be controlled by adjusting the ratio of LA and GA.

In the present invention, the biocompatible polymer nanoparticles may be chitosan-heparin nanocomplexes. The chitosan-heparin nanocomplex may form a complex through an ionic bond (i.e., ionic crosslinking) between the amine group of chitosan and the sulfate group of heparin. This nanocomplex may be maintained by electrostatic interaction between chitosan, which has a positive charge, and heparin, which has a negative charge.

According to one exemplary embodiment, the chitosan-heparin nanocomplex may have an average size of 200 to 1,000 nm. In the present invention, the “average size” may mean an average diameter of the chitosan-heparin nanocomplex in an aqueous medium. The average size can be measured through the method in the experimental example below. The average size may vary depending on the molecular weights of chitosan and heparin, the degree of deacetylation of chitosan, and the concentration and ratio of chitosan and heparin.

According to one exemplary embodiment, the chitosan-heparin nanocomplex may have a surface charge, which may vary depending on the composition ratio of chitosan and heparin. The positive charge is due to the amine group of chitosan, and the negative charge is due to the carboxyl and/or sulfate group of heparin.

According to one exemplary embodiment, the surface of the chitosan-heparin nanocomplex may exhibit a positive charge. The chitosan-heparin nanocomplex may bind to urease through its positive charge.

According to one exemplary embodiment, the ratio (volume ratio) of chitosan and heparin may be in the range of 1:0.25 to 0.3, specifically 1:0.25. Within the above content range, a nanocomplex whose surface has a positive charge may be produced, and binding with urease and encapsulating with a STING agonist may be easily achieved.

Also, in the present invention, the biocompatible polymer nanoparticles may be PLGA nanoparticles.

The surfaces of the PLGA nanoparticles may be modified with an amine group.

The PLGA nanoparticles may have an average size of 200 to 1,000 nm.

According to one exemplary embodiment, the surfaces of the biocompatible polymer nanoparticles may bind to urease which is a biological enzyme. Specifically, an amine group located on the surfaces of the biocompatible polymer nanoparticles may form bonds through urease and a dialdehyde compound.

The urease is an enzyme that hydrolyzes urea. The urease may act as an engine to move the nanomotor while decomposing urea present in a high concentration in the bladder, and is also biocompatible. Urea may be decomposed into ammonia and carbon dioxide by the urease.

The dialdehyde compound refers to a compound including two aldehyde groups in its structure. As such a dialdehyde compound, one or more selected from the group consisting of glutaraldehyde, glyoxal, and succinaldehyde may be used. Specifically, glutaraldehyde may be used.

According to one exemplary embodiment, the amine group of urease may react with one amine group of the dialdehyde compound to form a —C—N— bond in which the imine bond is reduced through reductive amination. Also, other amine groups of the dialdehyde compound may react with amine groups on the surfaces of the biocompatible polymer nanoparticles to form a —C—N— bond in which the imine bond is reduced through a reduction reaction.

According to one exemplary embodiment, the chitosan-heparin nanomotor may form a bond between the amine group of urease and the amine group of chitosan using glutaraldehyde as a linker. Also, according to one exemplary embodiment, the PLGA nanomotor may form a bond between the amine group of urease and the amine group on the surfaces of the PLGA nanoparticles using glutaraldehyde as a linker.

According to one exemplary embodiment, the content of urease may vary depending on the number of amine groups on the surfaces of biocompatible polymer nanoparticles.

In the present invention, the biocompatible polymer nanomotor may generate gas through the action of urease to induce self-propulsion.

Urease may decompose the urea in a urea environment to generate carbon dioxide, and the nanomotor may be propelled and moved through the generated carbon dioxide. Through this, the nanomotor may attach to the mucosa such as the bladder wall and the like, and also penetrate into the mucosa. Therefore, the biocompatible polymer nanomotor may also be expressed as a urease-bound (or propelled) biocompatible polymer nanomotor.

According to one exemplary embodiment, the biocompatible polymer nanomotor may have a size of 200 to 1,000 nm. Within the above size range, the biocompatible polymer nanomotor has the advantage of being easy to attach to and penetrate into the living body. When the size is too small, the propulsive force as a nanomotor may not be obtained. On the other hand, when the size is too large, there is a risk that a biological penetrating force may be reduced.

The biocompatible polymer nanomotor of the present invention may further include a drug encapsulated into the interior of the biocompatible polymer nanoparticles, and the drug may be a STING agonist.

The STING agonist may recognize cyclic dinucleotides (CDNs) to activate the type I pathway, thereby enhancing the innate immune response and thus the adaptive T cell response. The STING agonist may exhibit an anti-tumor effect by direct injection into the tumor.

This STING agonist has a negative charge and may react with the biocompatible polymer nanoparticles so that the STING agonist can be encapsulated into the interior of the nanoparticles. The STING agonist has a drawback in that its function may deteriorate when exposed to urine for a long period of time. Therefore, in the present invention, the STING agonist may be encapsulated into the interior of the nanoparticles to prevent exposure to the outside. Since the nanomotor may penetrate the bladder mucosa covered with the glycosaminoglycan layer, the cell delivery ability of the STING agonist may be further improved.

According to one exemplary embodiment, the STING agonist may be one or more selected from the group consisting of ADU-S100, MK-1454, MK-2118, SB11285, GSK3745417, BMS-986301, E7765, TAK-676, SNX-281, and SYNB1891.

According to one exemplary embodiment, the content of the STING agonist may be 1:0.1 to 0.2 relative to the content (weight) of the nanoparticles. In particular, when the chitosan-heparin nanocomplex is used, the content (weight) of chitosan may be 1:0.1 to 0.2. Within the above content range, the STING agonist is encapsulated into the interior of the biocompatible polymer nanoparticles, thereby exhibiting the effect of the STING agonist itself.

In the present invention, the biocompatible polymer nanomotor may further include a drug. At this time, the drug may form a bond with the surfaces of the biocompatible polymer nanoparticles.

The type of drug is not particularly limited, and anti-cancer drugs may be used in the present invention. One or more selected from the group consisting of paclitaxel, taxotere, adriamycin, endostatin, angiostatin, mitomycin, bleomycin, cisplatin, carboplatin, doxorubicin, daunorubicin, idarubicin, 5-fluorouracil and methotrexate, actinomycin-D may be used as the anti-cancer drug.

According to one exemplary embodiment, the biocompatible polymer nanomotor may be used to treat bladder diseases. At this time, the type of bladder disease is not particularly limited and, for example, may be selected from the group consisting of overactive bladder, interstitial cystitis, and bladder cancer.

In general, the treatment of bladder diseases is achieved by a method of injecting a drug into the bladder using a catheter. In this case, the method has the disadvantage that the drug does not attach well to the bladder wall and is washed away by frequent urination, thereby reducing the sustainability of the drug effect. In the present invention, when the biocompatible polymer nanomotor is used so that the biocompatible polymer nanomotor can be injected into the bladder, the nanomotor may be propelled by the high concentration of urea in the bladder, and may efficiently penetrate the mucosal layer of the bladder wall. Also, the biocompatible polymer nanomotor may remain in the bladder wall after urination. The improved penetration and retention of such a nanomotor suggests that the nanomotor may be used as a new method for treating various bladder diseases.

Also, the present invention relates to a method for producing the above-described biocompatible polymer nanomotor.

The biocompatible polymer nanomotor according to the present invention may include producing urease-bound biocompatible polymer nanoparticles by binding urease to the surfaces of biocompatible polymer nanoparticles.

In the present invention, the production of the urease-bound biocompatible polymer nanoparticles is a process of binding urease to the surfaces of the biocompatible polymer nanoparticles, that is, a process of producing a biocompatible polymer nanomotor.

According to one exemplary embodiment, the step may include allowing urease to react with an aqueous solution of a dialdehyde compound to prepare activated urease, and then adding the activated urease to an aqueous solution of biocompatible polymer nanoparticles.

Through the above steps, the dialdehyde compound may be used as a linker to form a bond between the amine group of urease and the amine group on the surfaces of the biocompatible polymer nanoparticles.

According to one exemplary embodiment, the reaction of the activated urease may be performed at 20 to 30° C. or room temperature for 30 minutes to 3 hours or 1 to 2 hours.

According to one exemplary embodiment, the biocompatible polymer nanomotor may have a size of 200 to 1,000 nm.

In the present invention, when the chitosan-heparin nanocomplex is used as the biocompatible polymer nanoparticles, the chitosan-heparin nanomotor may be produced by:
producing a chitosan-heparin nanocomplex by ionically bonding chitosan and heparin; and
binding urease to the surface of the chitosan-heparin nanocomplex to produce a urease-bound chitosan-heparin nanocomplex.

The step of producing of the chitosan-heparin nanocomplex is a process of producing a nanocomplex by ionically bonding chitosan and heparin.

In the above step, a nanocomplex may be formed by mixing a heparin solution and an aqueous chitosan solution. Specifically, a nanocomplex may be formed by slowly adding a heparin solution to an aqueous chitosan solution in a drop-by-drop manner.

Since chitosan has a positive charge and heparin has a negative charge, the chitosan and heparin may form a spherical nanocomplex via an ionic bond.

According to one exemplary embodiment, the ratio (volume ratio) of chitosan and heparin may be in the range of 1:0.25 to 0.3, specifically 1:0.25. Within the above content range, a nanocomplex whose surface has a positive charge may be produced, and encapsulating of the STING agonist may be easily performed.

According to one exemplary embodiment, the chitosan-heparin nanocomplex may have an average size of 200 to 1,000 nm.

Also, in the present invention, when PLGA nanoparticles are used as the biocompatible polymer nanoparticles, the PLGA nanomotor may be produced by binding urease to the surfaces of the PLGA nanoparticles, which have an amine group attached to the surfaces thereof, to produce urease-bound PLGA nanoparticles.

The production method of the present invention may further include encapsulating a STING agonist into the interior of the biocompatible polymer nanoparticles. The above step may be performed before allowing the urease to react with the biocompatible polymer nanoparticles.

According to one exemplary embodiment, the above step may be performed by adding an aqueous solution of the STING agonist to an aqueous solution including the biocompatible polymer nanoparticles. Also, the reaction can be performed using ultrasonic dispersion.

According to one exemplary embodiment, the content of the STING agonist may range from 1:0.1 to 0.2 relative to the content (weight) of the biocompatible polymer nanoparticles.

The production method of the present invention may further include binding a drug to the surfaces of the biocompatible polymer nanoparticles. At this time, the binding of the drug to the nanoparticles may be performed after the binding of the urease, but the drug may be bound first before binding the urease.

According to one exemplary embodiment, any of the above-described types of drugs may be used.

In addition, the present invention relates to a use of the biocompatible polymer nanomotor as described above.

The biocompatible polymer nanomotor according to the present invention may be used as a carrier for a drug delivery system. Also, the biocompatible polymer nanomotor may be coated on medical equipment such as a catheter, and then injected into the living body.

The biocompatible polymer nanomotor may include a drug, and the drug may be delivered into the living body through the mucosa by means of the propulsion of the nanomotor. At this time, any of the above-described types of drugs may be used.

The biocompatible polymer nanomotor of the present invention may be used to treat various diseases depending on the types of drugs. Specifically, the biocompatible polymer nanomotor may be used to treat a bladder disease. The bladder disease may be selected from the group consisting of overactive bladder, interstitial cystitis, and bladder cancer. Also, the biocompatible polymer nanomotor may be used for immunotherapy of bladder cancer.

Hereinafter, the present invention will be described in detail with reference to the following examples. However, it should be understood that the following examples are merely provided to illustrate the present invention, and are not intended to limit the scope of the present invention.

### MODE FOR INVENTION

### Examples

### <Reference> Statistical Analysis

Statistical analysis was performed by a t-test using SigmaPlot10.0 software.

Values of *P<0.05, **P<0.01, ***P<0.005, and ****P<0.001 were considered statistically significant. Data is expressed as mean±standard deviation (SD) from several individual experiments. All the experiments were performed in triplicate and 20 nanomotors were measured for each group.

### Example 1: Synthesis of STING Agonist-Encapsulated Chitosan-Heparin Nanomotor

### (1) Synthesis of STING Agonist-Encapsulated Chitosan-Heparin Nanocomplex (NC)

0.4, 0.5, and 0.6 mL of a heparin solution (1 mg/mL) was slowly added in a drop-by-drop manner to 2 mL of an aqueous chitosan solution (1 mg/mL) to form a chitosan-heparin nanocomplex.

Next, 0.1 or 0.2 mL of a STING agonist (ADU-S100) was slowly added to an aqueous chitosan-heparin solution, while the aqueous solution was subjected to tip sonication, to encapsulat the nanocomplex. To remove excess STING agonist, the nanocomplex was dialyzed in water for 3 days.

### (2) Synthesis of STING Agonist-Encapsulated Chitosan-Heparin Nanomotor

1 mL of urease (2 mg/mL) was reacted with 3 mL of an aqueous glutaraldehyde solution (2.5%). To remove unreacted glutaraldehyde, the nanocomplex was dialyzed for 3 days.

Next, 1 mL of the activated urease was added to the aqueous nanocomplex solution synthesized in (1), and reacted for an hour. Thereafter, the chitosan-heparin nanomotor solution was washed three times with PBS, and then centrifuged (4,000 rpm for 5 minutes).

Hereinafter, the chitosan-heparin nanocomplex is expressed as a nanocomplex, and the chitosan-heparin nanomotor is expressed as a nanomotor. Also, the STING agonist-encapsulated chitosan-heparin nanocomplex is expressed as a STING@nanocomplex, and the STING agonist-encapsulated chitosan-heparin nanomotor is expressed as a STING@nanomotor.

In the present invention, FIG. TA is a schematic diagram showing a method for producing a STING agonist-encapsulated chitosan-heparin nanomotor (STING@nanomotor).

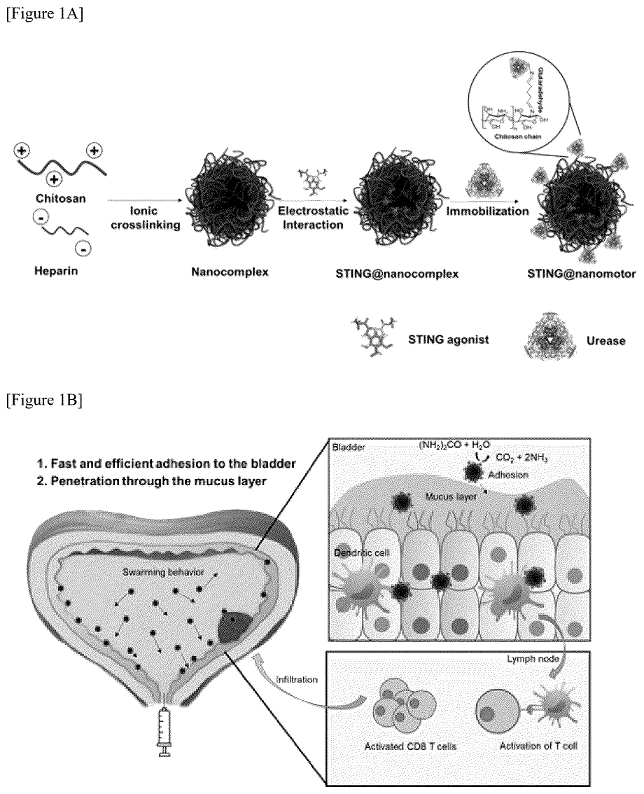

Also, FIG. 1B is a schematic diagram of the prepared STING agonist-encapsulated chitosan-heparin nanomotor's penetration into the bladder wall and activation of the immune response of dendritic cells.

As shown in FIG. 1, the STING@nanomotor may be designed and produced by a three-step process. First, chitosan and heparin polymers are ionically crosslinked to form a nanocomplex. Next, the STING agonist is encapsulated through electrostatic interaction. Finally, the amine group of urease and the amine group of chitosan are bound via a glutaraldehyde linker.

### Experimental Example 1: Characterization of STING Agonist-Encapsulated Chitosan-Heparin Nanomotor

### (1) Method

The characteristic shape of the STING agonist-encapsulated chitosan-heparin nanomotor produced in Example 1 was confirmed by transmission electron microscopy (TEM), and the size and zeta potential were measured using dynamic light scattering (DLS). The concentration of urease present on the surface of the nanomotor was measured using a Bradford protein analysis kit. Also, the enzymatic activity of urease bound to the nanocomplex was evaluated using a commercial kit to determine the concentration of ammonia produced by Berthelot's method. At this time, the concentration of the nanomotor was 0.5 mg/mL, and this experiment was performed according to the manufacturer's instructions. The release experiment of the STING agonist was performed in a PBS solution at 37° C. for 60 hours. The release of the STING agonist was determined using ultraviolet-visible spectroscopy (UV-Vis spectroscopy).

### (2) Results

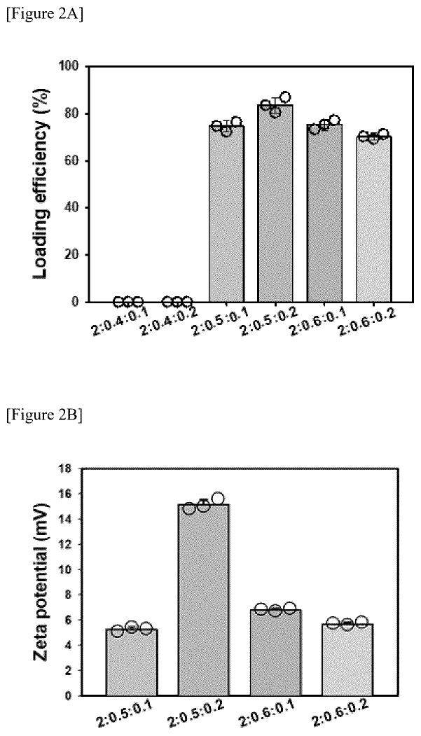

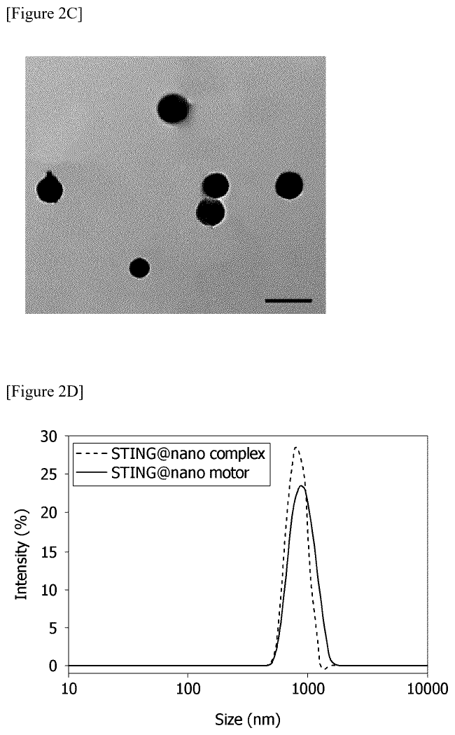

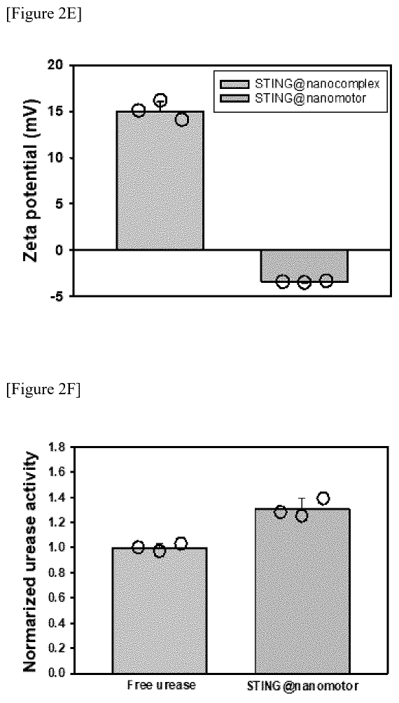

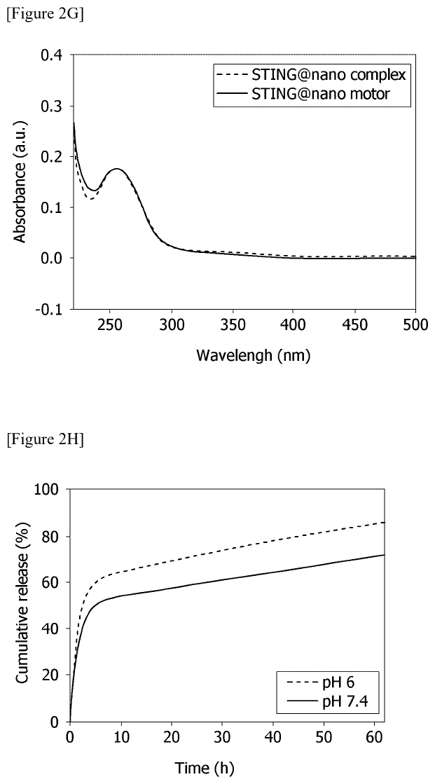

FIG. 2A shows the encapsulating efficiency of the STING agonist in the chitosan-heparin nanocomplex.

The STING agonist has anionic properties at neutral pH. Also, as shown in the drawing, it can be confirmed that the STING agonist is encapsulated into the chitosan-heparin nanocomplex with an efficiency of 70 to 83.5%.

FIG. 2B shows the zeta potential of the STING agonist-encapsulated chitosan-heparin nanocomplex.

As shown in the drawing, it can be confirmed that the STING agonist-encapsulated chitosan-heparin nanocomplex has cationic properties.

FIGS. 2C and 2D show the TEM and DLS images of the STING agonist-encapsulated chitosan-heparin nanomotor.

As shown in the drawing, it can be confirmed that the STING agonist-encapsulated chitosan-heparin nanomotor has a size of 200 to 1,000 nm.

FIG. 2E shows the zeta potential of the STING agonist-encapsulated chitosan-heparin nanocomplex before and after the attachment of urease, and FIG. 2F shows the results of the activity efficiency comparison of the urease-free STING agonist-encapsulated chitosan-heparin nanocomplex and urease-bound STING agonist-encapsulated chitosan-heparin nanocomplex (i.e., STING@nanomotor).

As shown in the drawing, the STING@nanomotor may have anionic properties in water due to the attachment of urease. Also, it can be seen that the efficiency of the enzyme-bound nanocomplex increases by 1 to 1.5 times compared to the same amount of the enzyme-free nanocomplex. This means that because urease is unstable in an aqueous solution, the urease is entangled, thereby lowering enzymatic activity, whereas the urease located on the surface of the nanomotor maintains high stability in an aqueous solution to preserve enzymatic activity.

Also, FIG. 2G shows the absorbance of the STING@nanocomplex and the STING@nanomotor, and FIG. 2H shows the release experiment results of the STING agonist in neutral and slightly acidic conditions.

As shown in the drawing, it can be seen that the STING agonist is slowly released from the nanomotor for 60 hours in neutral and slightly acidic conditions.

### Experimental Example 2: Analysis of Inflow and Activation of STING Agonist-Encapsulated Chitosan-Heparin Nanomotor into Dendritic Cells

### (1) Method

Murine dendritic cells (JAWS II) that were previously treated with 10 μg/mL of a chlorpromazine solution and 70 μg/mL of a genistein solution to block receptors on the cell surface were cultured in an alpha MEM medium. Thereafter, an FITC fluorophore-labeled STING agonist-encapsulated chitosan-heparin nanomotor was co-cultured for 2 hours. For the STING activation experiment, dendritic cells were cultured together with PBS, STING, the nanomotor, and the STING@nanomotor for 12 hours, and the activity of dendritic cells was measured through CD86 and CD40.

### (2) Results

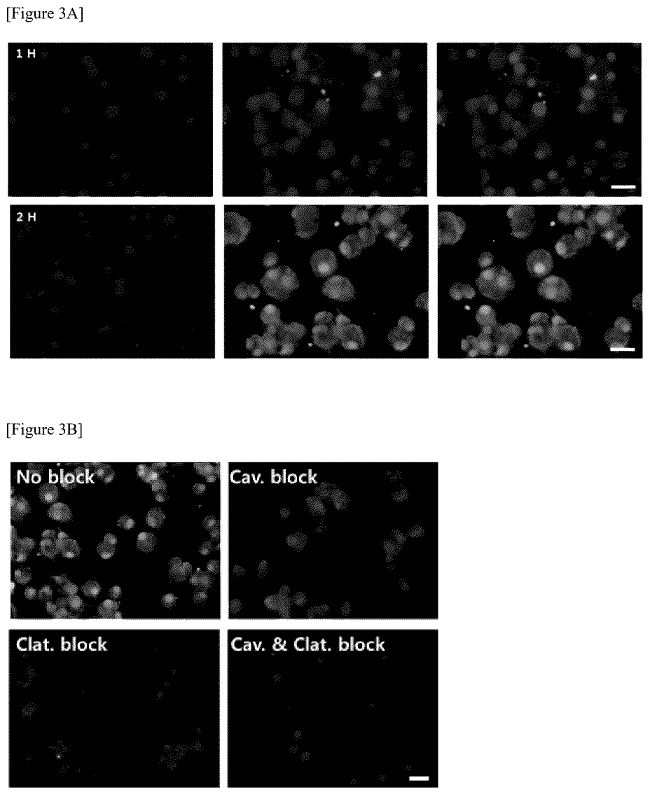

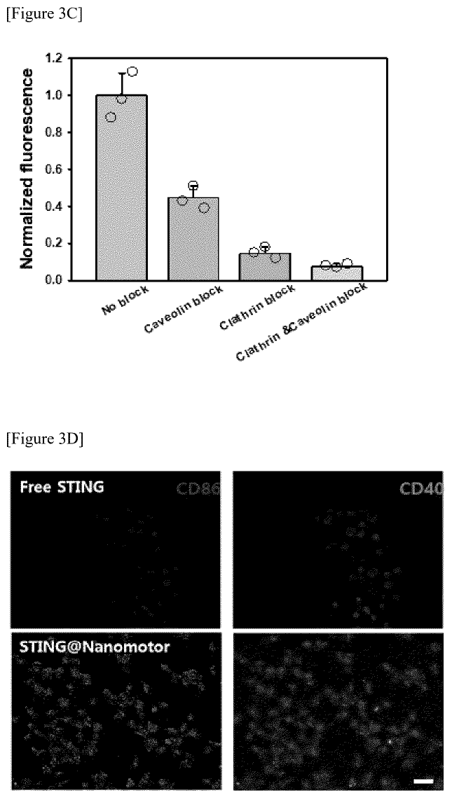

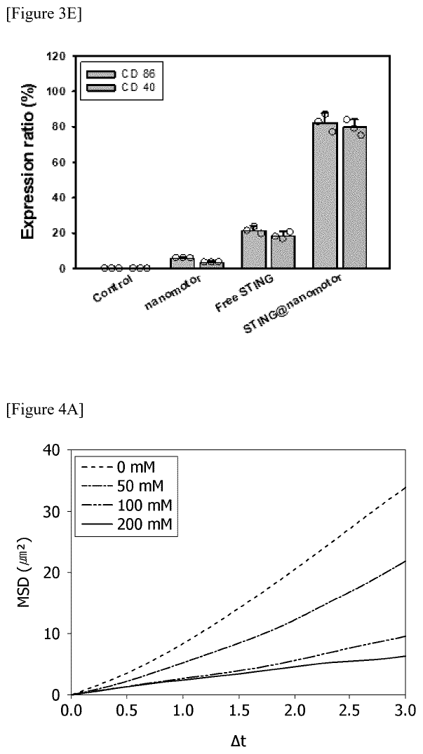

FIG. 3A is a fluorescent image of dendritic cells, showing the presence or absence of STING@nanomotor entering the dendritic cells, and FIGS. 3B and 3C show an inflow path mechanism of the STING@nanomotor by blocking various receptors on the surfaces of dendritic cells and then culturing the receptors with the STING@nanomotor. FIGS. 3D and 3E show the activation of dendritic cells by the STING@nanomotor.

As shown in FIG. 3A, the STING@nanomotor may be introduced into dendritic cells, and it can be seen that a greater amount of the STING@nanomotor is introduced over time. Also, as shown in FIGS. 3B and 3C, the inflow path mechanism was confirmed through changes in an inflow amount of the STING@nanomotor. As a result, it was confirmed that the inflow mechanism was mostly associated with caveolin and clathrin receptor-related cell inflow.

Also, as shown in FIGS. 3D and 3E, the STING@nanomotor may be introduced into dendritic cells to activate the dendritic cells, and the STING agonist may be released from the nanomotor to activate the dendritic cells. The activity of dendritic cells was analyzed through the expression of CD 80 and CD 40 on the surfaces of dendritic cells. As a result of confirming activity through CD 80 and CD 40 target molecules, it was confirmed that the nanomotor has activities of 82.1 and 79.6% for the CD 80 and CD 40 target molecules, respectively.

### Experimental Example 3: Video Recording and Nanomotor Motility Analysis

### (1) Method

The movement of STING@nanomotor was observed and recorded on a video using an optical microscope.

An aqueous solution sample of the nanomotor was placed on a glass slide and mixed with various aqueous solutions of urea (concentrations of 0, 50, 100, and 200 mM). The movement of STING@nanomotor was recorded for 15 seconds at a frame rate of 40 fps. Twenty or more STING@nanomotors per condition were analyzed, and the tracking path, mean square displacement (MSD), and speed of the STING@nanomotor were automatically analyzed using a Python program. Thereafter, the speed was obtained by fitting the MSD data to Equation 1 below.

$$MSD ( Δ t ) = {( V Δ t )}^{2} + 4 D Δ t \qquad 〈 Equation 1 〉$$

wherein V represents the speed, De represents the effective diffusion coefficient, and Δt represents the time interval.

The cluster motility of the STING@nanomotor was analyzed using an optical microscope with 2.5 magnifications.

5 μL of the STING@nanomotor solution was dropped into a petri dish containing 2 mL of PBS and an aqueous urea solution, and the motility of the STING@nanomotor was measured for 90 seconds.

### (2) Results

STING@nanomotor driven by urease converts urea into ammonia and carbon dioxide, as shown in Equation 2 below.

(NH₂)₂CO+H₂O→CO₂+2NH₃ <Equation 2>

Although the geometric asymmetry in a synthetic motor has been considered an important requirement for generating propulsion, recent studies have shown that only an unbalanced distribution of molecules bound to the surface of the nanomotor is sufficient for propulsion of the synthetic motor driven by the biocatalyzed transformation of the enzyme. The kinetic profile of STING@nanomotor was evaluated at urea concentrations of 0, 50, 100 and 200 mM.

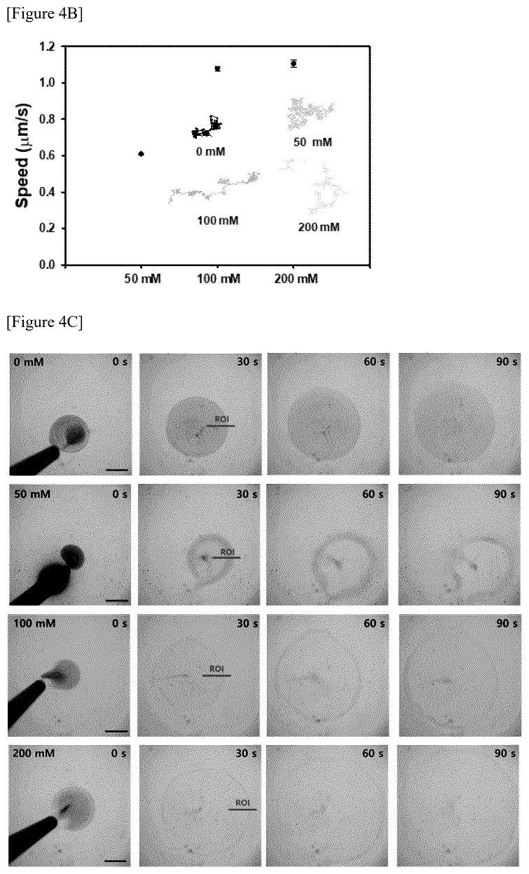

FIGS. 4A and 4B show the MSD data and speed. In FIG. 4, the tracking trajectory was recorded for 15 seconds at 40 frames per second. Also, the speed and mean square displacement (MSD) were calculated from the tracked trajectory.

When there was no urea, the STING@nanomotor showed Brownian motion and no directionality. However, it can be seen that after urea was added (50, 100, and 200 mM), the STING@nanomotor showed both improved speed and directionality. Also, it can be seen that the MSD increases in a non-linear manner and has a higher rate of change as the concentration of urea increases.

Also, FIG. 4C shows the diffusion area of the STING@nanomotor cluster for 90 seconds.

The STING@nanomotor cluster hardly expanded when there was no urea. On the other hand, it can be seen that the STING@nanomotor cluster dropped into the urea-containing solution expanded rapidly and spread to a larger area after 90 seconds. Also, it can be confirmed that this area is related to the concentration of urea. From the above results, it can be confirmed whether the STING@nanomotor cluster effectively moves within the bladder having a high concentration of urea.

### Experimental Example 4: Bio-Imaging

### (1) Method

To investigate the nanomotor's ability to penetrate the bladder wall and remain in the bladder, the surface of the nanomotor was labeled with a fluorescent material. As a control, a nanocomplex having no propulsion ability was also labeled with a fluorescent material.

To label the nanocomplex and the nanomotor with an FITC fluorescent dye, 100 μL of an FITC solution (1 mM) was added to 2 mL of aqueous nanocomplex and nanomotor solutions, respectively. The reaction was carried out by incubating the mixture at room temperature for 12 hours. Then, the labeled nanocomplex and nanomotor solutions were centrifuged to remove unreacted FITC molecules.

Balb/c female mice were randomly divided into two groups (nanomotor and nanocomplex) (n=3), and then anesthetized through inhalation anesthesia. A 50 μL suspension of the fluorescently labeled sample was administered into the bladder via a catheter. After administration, the mice were sacrificed, and the intact bladder was incised and cut to observe the bladder wall. Thereafter, the tissue was rinsed with PBS, flattened, and visualized using two-photon fluorescence microscopy. Images were collected as Z-stacks (xyz, 400 Hz) at 512×512 pixels and analyzed using Leica's LAS AF Lite 2.6.1. To confirm the retention of the nanomotor in the bladder, the bladders were extracted from sacrificed mice 12 hours later, excised, and fixed in 4% paraformaldehyde. The fixed bladders were embedded in paraffin blocks, and cut into 4-μm-thick sections for H&E staining. The stained parts were observed under an optical microscope. Also, biological images were obtained through IVIS imaging 12 hours later.

### (2) Results

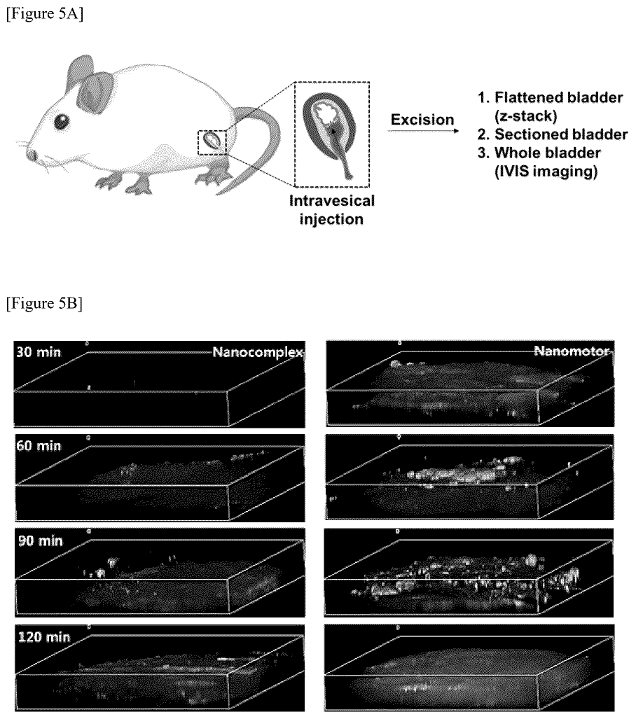

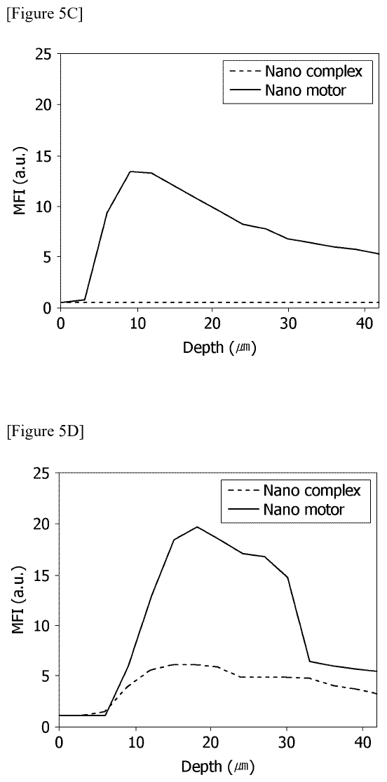

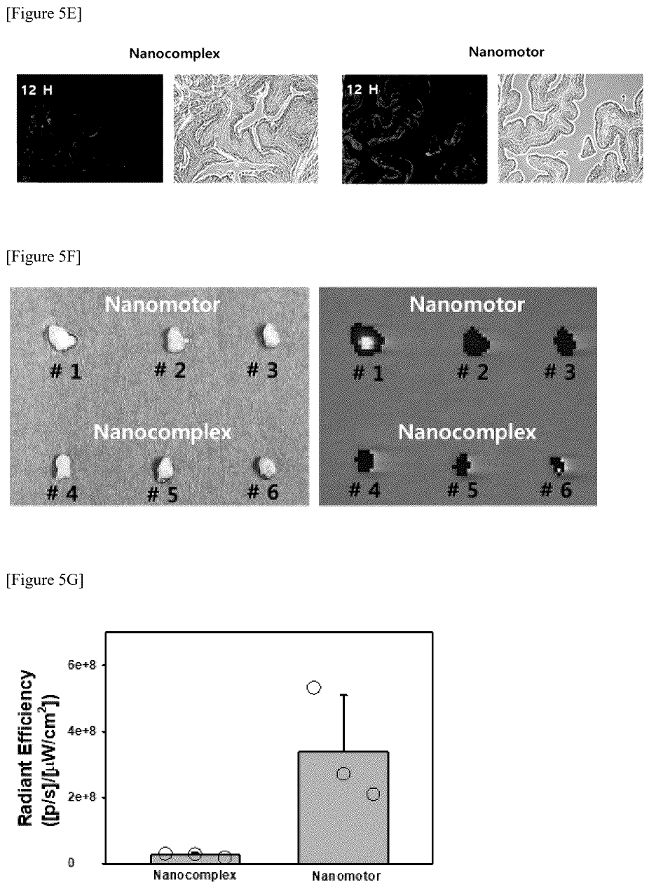

The results are shown in FIG. 5. FIG. 5A shows a schematic bio-imaging diagram for confirming penetration and retention efficacy in the bladder.

Biological images are measured using three methods: the degree of penetration into the bladder wall was confirmed through two-photon microscopy, and fluorescence images were obtained through a cross-section of bladder tissue. Also, the sample retention in the entire bladder tissue was confirmed through IVIS imaging.

FIGS. 5B, 5C and 5D show the bladder tissue and the fluorescence intensity according to the penetration depth measured by two-photon microscopy.

As shown in the drawings, it can be seen that the penetration into the bladder wall for 120 minutes was significantly low in the case of the nanocomplex group, compared to that of the nanomotor group.

FIG. 5E shows the fluorescence image of the bladder 12 hours after the injection of the nanocomplex and nanomotor.

As shown in the drawing, it can be seen through the cross section of bladder tissue extracted after 12 hours that the nanomotor group has a much higher level of retention than the nanocomplex group.

Also, FIGS. 5F and 5G show the entire IVIS bladder images and the fluorescence intensity obtained through IVIS imaging.

As shown in the drawings, it can be seen that the higher fluorescence intensity was observed in the nanomotor group compared to the nanocomplex group. Based on these results, it was confirmed that the nanomotor group had higher penetration and retention abilities after intravesical injection.

### Experimental Example 5: Creation of Bladder Cancer Model and Confirmation of Bladder Cancer Therapeutic Efficacy

### (1) Method

A bladder cancer model was created using 8-week-old C57BL/6J mice. MB49 cells were used to create a bladder cancer model. First, 100 μL of HCl was injected into the bladder for 3 minutes in order to improve the implantation efficacy of cancer cells. Thereafter, 100 μL of a PBS solution containing 1×10⁶ cells was injected into the bladder through a 24 G angiocath catheter. To confirm the anti-cancer effect, STING (10 μg/100 μL), the nanomotor, and the STING agonist-encapsulated nanocomplex (STING@nanocomplex) (10 μg STING/100 μL), and the STING agonist-encapsulated nanomotor (STING@nanomotor) (10 μg STING/100 μL) were injected into the bladder.

Bladders were pulverized for RNA extraction and total RNA was reverse transcribed into cDNA. Cytokines were confirmed through a real-time polymerase chain reaction (RT-PCR). GAPDH was used as a reference gene. PCR primer sequences are listed in Table 1 below.

TABLE 1

| | | | |
|---|---|---|---|
| GAPDH | Forward | AGGTCGGTGTGAACGGATTTG | SEQ ID |
| | | | NO: 1 |
| | Reverse | TGTAGACCATGTAGTTGAGGTCA | SEQ ID |
| | | | NO: 2 |
| IL-1βq | Forward | GCAACTGTTCCTGAACTCAACT | SEQ ID |
| | | | NO: 3 |
| | Reverse | ATCTTTTGGGGTCCGTCAACT | SEQ ID |
| | | | NO: 4 |
| IL-6 | Forward | TGGGGCTCTTCAAAAGCTCC | SEQ ID |
| | | | NO: 5 |
| | Reverse | AGGAACTATCACCGGATCTTCAA | SEQ ID |
| | | | NO: 6 |
| IFN β | Forward | CAGCTCCAAGAAAGGACGAAC | SEQ ID |
| | | | NO: 7 |
| | Reverse | GGCAGTGTAACTCTTCTGCAT | SEQ ID |
| | | | NO: 8 |
| CXCL10 | Forward | CCAAGTGCTGCCGTCATTTTC | SEQ ID |
| | | | NO: 9 |
| | Reverse | GGCTCGCAGGGATGATTTCAA | SEQ ID |
| | | | NO: 10 |

### (2) Results

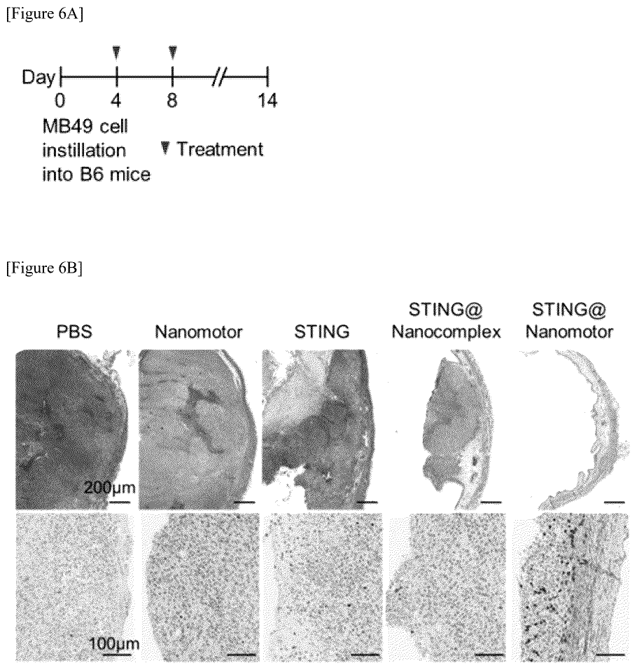

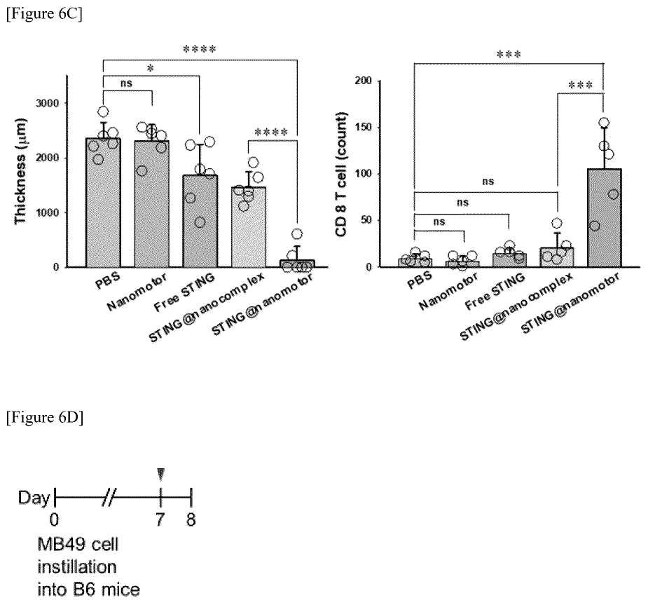

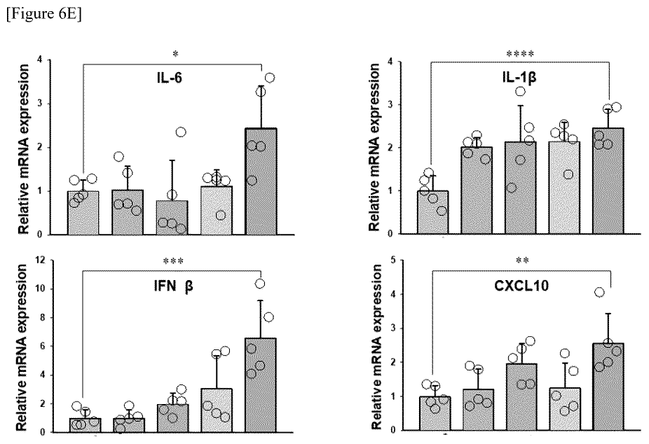

FIG. 6A shows the schedule for the production and treatment of a bladder cancer model In this experimental example, treatment was performed 4 and 8 days after bladder cancer cell injection, and bladder tissue was removed 2 weeks later to confirm the anti-cancer effect.

FIGS. 6B, 6C and 6D show a cross-sectional view of the bladder, the thickness of cancer tissue, and the number of immune cells after 2 weeks, respectively.

As shown in the drawings, the highest anti-cancer effect may be observed in the STING@nanomotor group, which is the result of an increase in the number of immune cells. Also, it can be confirmed that T cells having cytotoxicity are introduced into the bladder to effectively kill bladder cancer.

Also, FIG. 6E shows the results of comparison of immune-related mRNA expression. IL-6, IL-1B, IFN B, and CXCL10 were measured, which are mRNAs associated with immune responses.

As shown in the drawing, it can be seen that the highest mRNA expression is observed in the STING@nanomotor group.

### Example 2: Synthesis of STING Agonist-Encapsulated PLGA Nanomotor

### (1) Synthesis of PLGA Nanoparticles

A PLGA solution was prepared by dissolving 2% by weight of PLGA in methylene chloride, and a PVA solution was prepared by dissolving polyvinyl alcohol) (PVA) in distilled water. The PLGA solution was dropped little by little into the PVA solution. Thereafter, ultrasonic waves were applied for 5 minutes to form particles, and an aqueous solution in which the particles were formed was evaporated to obtain only the particles.

### (2) Synthesis of STING Agonist-Encapsulated PLGA Nanoparticles

Amine group-bound PLGA nanoparticles were dissolved in 0.5 mL of a dichloromethane organic solvent (20 mg/mL). The solution was added in a drop-by-drop manner to 2 mL of an aqueous solution containing polyvinyl alcohol (10 mg/mL) and the STING agonist (2 mg/mL). Particle homogenization was performed using a tip sonicator (preparation of particle suspension).

The particle suspension was added in a drop-by-drop manner to 40 mL of water, and the reaction was carried out in a hood for 3 hours to volatilize the organic solvent. It was separated through centrifugation (8000 rpm for 5 minutes).

### (3) Synthesis of STING Agonist-Encapsulated PLGA Nanomotor

1 mL of urease (2 mg/mL) was allowed to react with 3 mL of an aqueous glutaraldehyde solution (2.5%). To remove unreacted glutaraldehyde, the reaction mixture was dialyzed for 3 days.

Next, 1 mL of the activated urease was added to the aqueous solution of nanoparticles synthesized in (2) and reacted for an hour. Thereafter, the PLGA nanomotor solution was washed three times with PBS, and then centrifuged (8000 rpm for 5 minutes).

### INDUSTRIAL APPLICABILITY

The biocompatible polymer nanomotor according to the present invention can penetrate deeply through the mucosal layer in the bladder and remain in the bladder wall for a long time to treat bladder cancer.

In particular, the chitosan-heparin nanomotor can move autonomously in the presence of urea and reach the bladder wall so that the chitosan-heparin nanomotor can be effectively attached to the mucosal layer due to the chitosan on the surface of the nanomotor.

1. A biocompatible polymer nanomotor comprising:
biocompatible polymer nanoparticles; and
urease bound to the surfaces of the biocompatible polymer nanoparticles,
wherein the biocompatible polymer nanoparticles include one or more selected from the group consisting of chitosan, heparin, and poly(lactide-co-glycolide) (PLGA), and
the urease generates gas in the presence of urea to induce self-propulsion of the nanomotor.

2. The biocompatible polymer nanomotor of claim 1, wherein the biocompatible polymer nanoparticles are chitosan-heparin nanocomplexes.

3. The biocompatible polymer nanomotor of claim 2, wherein the chitosan-heparin nanocomplex is a complex formed via an ionic bond (i.e., ionic crosslinking) between an amine group of chitosan and a sulfate group of heparin.

4. The biocompatible polymer nanomotor of claim 1, wherein the biocompatible polymer nanoparticles are PLGA nanoparticles, and
the surfaces of the PLGA nanoparticles are modified with an amine group.

5. The biocompatible polymer nanomotor of claim 1, wherein a dialdehyde compound is used as a linker to form a bond between an amine group of the urease and an amine group on the surface of the biocompatible polymer.

6. The biocompatible polymer nanomotor of claim 1, wherein the biocompatible polymer nanomotor has a size of 200 to 1,000 nm.

7. The biocompatible polymer nanomotor of claim 1, further comprising a drug encapsulated inside the biocompatible polymer nanoparticles.

8. The biocompatible polymer nanomotor of claim 7, wherein the drug is a STING agonist.

9. The biocompatible polymer nanomotor of claim 1, further comprising a drug bound to the surfaces of the biocompatible polymer nanoparticles, and
the drug is one or more anti-cancer drug selected from the group consisting of paclitaxel, taxotere, adriamycin, endostatin, angiostatin, mitomycin, bleomycin, cisplatin, carboplatin, doxorubicin, daunorubicin, idarubicin, 5-fluorouracil, methotrexate, and actinomycin-D.

10. The biocompatible polymer nanomotor of claim 1, which is used for the treatment of one or more bladder diseases selected from the group consisting of overactive bladder, interstitial cystitis, and bladder cancer.

11. A method for producing a biocompatible polymer nanomotor according to claim 1, comprising:
producing urease-bound biocompatible polymer nanoparticles by binding urease to the surfaces of biocompatible polymer nanoparticles,
wherein the biocompatible polymer nanoparticles include one or more selected from the group consisting of chitosan, heparin, and poly(lactide-co-glycolide) (PLGA).

12. The method of claim 11, wherein the producing of the urease-bound biocompatible polymer nanoparticles includes forming a bond between an amine group on the surfaces of the biocompatible polymer nanoparticles and an amine group of the urease using a dialdehyde compound as a linker.

13. The method of claim 11, further comprising:
producing a chitosan-heparin nanocomplex by ionically bonding chitosan and heparin; and
binding urease to the surface of the chitosan-heparin nanocomplex to produce a urease-bound chitosan-heparin nanocomplex.

14. The method of claim 11, further comprising:
binding urease to the surfaces of the PLGA nanoparticles having amine groups bound to the surfaces thereof to produce urease-bound PLGA nanoparticles.

15. The method of claim 11, further comprising:
encapsulating the interior of the biocompatible polymer nanoparticles with a STING agonist.

16. A carrier for a drug delivery system comprising the biocompatible polymer nanomotor according to claim 1.
